(19) United States
(12) Patent Application Publication
(10) Pub. No.: US 2007/0036862 A1
(43) Pub. Date: Feb. 15, 2007

(54) Treatment with azetidinone-based cholesterol absorption inhibitors and omega-3 fatty acids and a combination product thereof

(76) Inventors: Roelof M. L. Rongen, Califon, NJ (US); Robert A. Shalwitz, Bexley, OH (US); George Bobotas, Tarpon Springs, FL (US); Abdel Fawzy, Easton, PA (US); Egil Bodd, Olso (NO)

Correspondence Address: ARENT FOX PLLC, 1050 CONNECTICUT AVENUE, N.W., SUITE 400, WASHINGTON, DC, 20036, US

(21) Appl. No.: 11/488,181

(22) Filed: Jul. 18, 2006

Related U.S. Application Data
(60) Provisional application No. 60/699,866, filed on Jul. 18, 2005.

(51) Int. Cl. A61K 31/397; A61K 9/24; A61K 31/202

(52) U.S. Cl. 424/472; 514/210.02; 514/560

(57) ABSTRACT

Combinations of one or more azetidinone-based cholesterol absorption inhibitors with mixtures of omega-3 fatty acids, methods of administering such combinations, and unit dosages of such combinations.

This is a nonprovisional application of provisional patent application No. 60/699,866, filed Jul. 18, 2005. The disclosure of the prior application is hereby incorporated by reference herein in its entirety.

### FIELD OF THE INVENTION

The present invention relates to combinations of one or more azetidinone-based cholesterol absorption inhibitors, preferably ezetimibe, with mixtures of omega-3 fatty acids that include eicosapentaenoic acid (EPA) and docosahexaenoic acid (DHA), preferably Omacor® omega-3 fatty acids, to methods of administering such combinations, and to unit dosages of such combinations. The present invention also relates to utilizing combinations of one or more azetidinone-based cholesterol absorption inhibitors with mixtures of omega-3 fatty acids for the treatment of patients with one or more of dyslipidemia and related conditions, renal disease, hypercholesterolemia, elevated total cholesterol (total-C), elevated low density lipoprotein cholesterol (LDL-C), and elevated apolipoprotein (Apo B), low high density lipoprotein cholesterol (HDL-C), elevated sitosterol, elevated campesterol, sitosterolemia, cholesterol-associated benign and malignant tumors and/or any other conditions that would benefit from treatment with such combinations. The present invention also relates to a single administration combination product of one or more azetidinone-based cholesterol absorption inhibitors and Omacor® omega-3 acids.

### BACKGROUND OF THE INVENTION

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (total-C), LDL-C, and apolipoprotein B (a membrane complex for LDL-C) promote human atherosclerosis and decreased levels of HDL-C and its transport complex, apolipoprotein A, which are associated with the development of atherosclerosis. Further, cardiovascular morbidity and mortality in humans can vary directly with the level of total-C and LDL-C and inversely with the level of HDL-C.

Azetidinone-based cholesterol absorption inhibitors are known (see for example Rosenblum, S. B., et al., J. Med. Chem., 41(6):973-80 (1998)). Azetidinone-based compounds can be inhibitors of cholesterol absorption (see Bioorg. Med. Chem., 7(10):2199-202 (1999)). One azetidinone-based compound is ezetimibe (1-(4-fluorophenyl)-(3R-)-[3-(4-fluorophenyl)-(3S)-hydroxypropyl]-(45)-(4-hydroxyphenyl)-2-azetidinone) (also known as SCH 58235 or ZETIA®) and its phenolic glucuronide, SCH60663 (see Br. J. Pharmacol., 129(8):1748-54 (2000)). U.S. Published Patent Application No. US 2004/0116358 A1 discloses compositions of ezetimibe and methods for the treatment of cholesterol-associated benign and malignant tumors.

Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

One form of omega-3 fatty acid is a concentrate of omega-3, long chain, polyunsaturated fatty acids from fish oil containing DHA and EPA and is sold under the trademark OMACOR®. Such a form of omega-3 fatty acid is described, for example, by U.S. Pat. Nos. 5,502,077, 5,656,667 and 5,698,594, each of which is incorporated herein by reference in their entireties.

U.S. Patent Application Publication No. 2006/0034815, which is incorporated herein by reference in its entirety, discloses a pharmaceutical composition comprising an omega-3 oil and one or more salts of a statin, wherein at least about 80 percent of the statin by weight is present as solid particles in heterogeneous suspension. In another embodiment, the publication provides a pharmaceutical composition comprising an omega-3 oil and one or more salts of a statin, wherein up to 15 percent of the amount of statin by weight is in solution while the amount of remaining statin is present in heterogeneous suspension.

### SUMMARY OF THE INVENTION

There is an unmet need in the art for the co-administration of one or more azetidinone-based cholesterol absorption inhibitors, preferably ezetimibe, with mixtures of omega-3 fatty acids that include eicosapentaenoic acid (EPA) and docosahexaenoic acid (DHA), preferably Omacor® omega-3 fatty acids. Further, there is an unmet need for a combination product that provides a single administration of omega-3 fatty acids (e.g., the Omacor® omega-3 acids) and one or more azetidinone-based cholesterol absorption inhibitors, for example, in a unit dosage. There is also an unmet need in the art for a method of administration of a single administration or unit dosage product. Moreover, there is an unmet need in the art for one or more azetidinone-based cholesterol absorption inhibitors and the Omacor® omega-3 acids, wherein one or more azetidinone-based cholesterol absorption inhibitors are combined with the Omacor® omega-3 acids to provide the specific therapeutic properties. There is a further need to provide a unit dosage of one or more azetidinone-based cholesterol absorption inhibitors and omega-3 fatty acids that can avoid significant degradation over time.

The present invention meets the unmet needs of the art, as well as others, by providing for concomitant co-administration, or an administration of a unit dosage of one or more azetidinone-based cholesterol absorption inhibitors, preferably ezetimibe, with mixtures of omega-3 fatty acids that include eicosapentaenoic acid (EPA) and docosahexaenoic acid (DHA), preferably Omacor® omega-3 fatty acids, that can provide an effective pharmaceutical treatment of one or more of dyslipidemia and related conditions, renal disease, hypercholesterolemia, elevated total cholesterol (total-C), elevated low density lipoprotein cholesterol (LDL-C), and elevated apolipoprotein (Apo B), low high density lipoprotein cholesterol (HDL-C), elevated sitosterol, elevated campesterol, sitosterolemia, cholesterol-associated benign and malignant tumors and/or any other conditions that would benefit from treatment with such combinations, for examples coronary heart disease, vascular disease, and related disorders, events, and/or symptoms.

Some embodiments of the present invention provide for a method of co-administering or utilizing a combination product of one or more azetidinone-based cholesterol absorption inhibitors, preferably ezetimibe, with mixtures of omega-3 fatty acids that include eicosapentaenoic acid (EPA) and docosahexaenoic acid (DHA), preferably Omacor® omega-3 fatty acids, in the treatment of one or more of dyslipidemia and related conditions, renal disease, hypercholesterolemia, elevated total cholesterol (total-C), elevated low density lipoprotein cholesterol (LDL-C), and elevated apolipoprotein (Apo B), low high density lipoprotein cholesterol (HDL-C), elevated sitosterol, elevated campesterol, sitosterolemia, cholesterol-associated benign and malignant tumors and/or any other conditions that would benefit from treatment with such combinations, as well as patients with hypertriglyceridemia, vascular disease, artherosclerotic disease and related conditions, patients in need of the prevention or reduction of cardiovascular and vascular events, and the reduction of triglyceride levels, insulin resistance, fasting glucose levels and postprandial glucose levels. Preferred embodiments include treatment of mixed dyslipidemia, combined hyperlipidemia, and reduction of non-HDL-C.

Other embodiments of the present invention are directed to a combination product, for example, a unit dosage, comprising one or more azetidinone-based cholesterol absorption inhibitors, preferably ezetimibe, with mixtures of omega-3 fatty acids that include eicosapentaenoic acid (EPA) and docosahexaenoic acid (DHA), preferably Omacor® omega-3 fatty acids. In one aspect of the embodiment, the combination product is used in the treatment of one or more of dyslipidemia and related conditions, renal disease, hypercholesterolemia, elevated total cholesterol (total-C), elevated low density lipoprotein cholesterol (LDL-C), and elevated apolipoprotein (Apo B), low high density lipoprotein cholesterol (HDL-C), elevated sitosterol, elevated campesterol, sitosterolemia, cholesterol-associated benign and malignant tumors and/or any other conditions that would benefit from treatment with such combinations, as well as patients with hypertriglyceridemia, vascular disease, artherosclerotic disease and related conditions, patients in need of the prevention or reduction of cardiovascular and vascular events, and the reduction of triglyceride levels, insulin resistance, fasting glucose levels and postprandial glucose levels. Preferred embodiments include treatment of mixed dyslipidemia, combined hyperlipidemia, and reduction of non-HDL-C.

Other features and advantages of the present invention will become apparent to those skilled in the art upon examination of the following or upon learning by practice of the invention.

### DESCRIPTION OF THE PREFERRED EMBODIMENTS

A particularly preferred azetidinone-based compound for use in compositions and methods of the present invention is ezetimibe or a stereoisomeric mixture thereof, diastereomerically enriched, diastereomerically pure, enantiomerically enriched or enantiomerically pure isomer thereof, or a prodrug of such compound, mixture or isomer thereof, or a pharmaceutically acceptable salt of the compound, mixture, isomer or prodrug. Another preferred azetidinone-based cholesterol absorption inhibitor is the phenolic glucuronide of ezetimibe or a stereoisomeric mixture thereof, diastereomerically enriched, diastereomerically pure, enantiomerically enriched or enantiomerically pure isomer thereof, or a prodrug of such compound, mixture or isomer thereof, or a pharmaceutically acceptable salt of the compound, mixture, isomer or prodrug. Two other ezetimibe related analogs and cholesterol absorption inhibitors for use in compositions and methods of the present invention, for example, are referred to in the literature as: 1) SCH 58053 or (+)-7-(4-chlorophenyl)-2-(4-flourophenyl)-7-hydroxy-3R-(4-hydroxyphenyl)-2-azaspiro[3,5]nonan-1-one) (see J. Lipid Res. 43:1864-1873(2002)) and 2) SCH 48461 or (3R)-3Phenylpropyl)-1,(4S)-bis(4-methoxyphenyl)-2-azetidinone (see J. Med. Chem., 41:973-980 (1998)).

Ezetimibe's mode of action involves the inhibition of cholesterol absorption and resorption in the intestinal tract. This mechanism of action also involves the increased excretions of cholesterol and its intestinal generated metabolites with the feces. This effect of ezetimibe results in lowered body cholesterol levels, increased cholesterol synthesis, and decreased triglyceride synthesis. The increased cholesterol synthesis initially provides for the maintenance of cholesterol levels in the circulation, levels that eventually decline as the inhibition of cholesterol absorption and resorption continues. The overall effect of drug action is the lowering of cholesterol levels in the circulation and tissues of the body.

The expression “prodrug” as used herein refers to compounds that are drug precursors which following administration, release the drug in vivo via chemical or physiological process (e.g., a prodrug on being brought to the physiological pH is converted to the desired drug form). Exemplary prodrugs upon cleavage release the corresponding free acid. For example, by means of hydrolyzable ester-forming residues of the compounds.

Compositions of the invention basically comprise an effective dose or a pharmaceutically effective amount or a therapeutically effective amount of an azetidinone based cholesterol absorption inhibitor, preferably ezetimibe and/or its phenolic glucuronide or at least one ezetimibe pharmacologically active analog.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated. The percentage by weight may be based on the free acid or ester forms, although it is preferably based on the ethyl ester form of the omega-3 fatty acids even if no other forms are utilized in accordance with the present invention.

The EPA:DHA ratio may be from 99:1 to 1:99, preferably 4:1 to 1:4, more preferably 3:1 to 1:3, most preferably 2:1 to 1:2. The omega-3 fatty acids may comprise pure EPA or pure DHA.

The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is the Omacor® omega-3 acid (K85EE, Pronova Biocare A.S., Lysaker, Norway) and preferably comprises the following characteristics (per dosage form):

| Test | Minimum Value | Maximum Value |
|---|---|---|
| Eicosapentaenoic acid C20:5 | 430 mg/g | 495 mg/g |
| Docosahexaenoic acid C22:6 | 347 mg/g | 403 mg/g |
| EPA and DHA | 800 mg/g | 880 mg/g |
| Total n-3 fatty acids | 90% (w/w) | |

The azetidinone-based cholesterol absorption inhibitors and/or omega-3 fatty acids may be administered by any means known in the art. Such modes include oral, rectal, nasal, topical (including buccal and sublingual) or parenteral (including subcutaneous, intramuscular, intravenous and intradermal) administration. These compositions are preferably orally administered.

The dosage of active ingredients in the compositions of this invention may be varied; however, it is necessary that the amount of the active ingredients be such that a suitable dosage form is obtained. The selected dosage depends upon the desired therapeutic effect, on the route of administration, and on the duration of the treatment. Compositions of some embodiments of the invention basically comprise an effective dose, a pharmaceutically effective amount, or a therapeutically effective amount of one or more azetidinone-based cholesterol absorption inhibitors.

The combination product of an azetidinone-based cholesterol absorption inhibitor, preferably ezetimibe, with mixtures of omega-3 fatty acids that include eicosapentaenoic acid (EPA) and docosahexaenoic acid (DHA), preferably Omacor® omega-3 fatty acids, may be administered in a capsule, a tablet, a powder that can be dispersed in a beverage, a liquid, a soft gelatin capsule or other convenient dosage form such as oral liquid in a capsule, as known in the art. In some embodiments, the capsule comprised of hard gelatin. The combination product may also be contained in a liquid suitable for injection or infusion.

The active ingredients of the present invention, an azetidinone-based cholesterol absorption inhibitor, preferably ezetimibe, and mixtures of omega-3 fatty acids that include eicosapentaenoic acid (EPA) and docosahexaenoic acid (DHA), preferably Omacor® omega-3 fatty acids, may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthum gum.

In most embodiments, excipients primarily include surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The omega-3 fatty acids can be administered in a daily amount of from about 0.1 g to about 10 g, more preferably about 0.5 g to about 8 g, and most preferably from about 0.75 g to about 4 g. Preferably, in the unit dosage form, the omega-3 fatty acids are present in an amount from about 0.1 g to about 2 g, preferably about 0.5 g to about 1.5 g, more preferably about 1 g.

In one embodiment of the present invention, the azetidinone-based cholesterol absorption inhibitor, preferably ezetimibe, can generally be administered in an amount from about 2 mg to 150 mg, more preferably from about 5 mg to about 100 mg, and even more preferably from about 10 mg to about 50 mg.

In some variations of the present invention, the combination of an azetidinone-based cholesterol absorption inhibitor, preferably ezetimibe, with mixtures of omega-3 fatty acids that include eicosapentaenoic acid (EPA) and docosahexaenoic acid (DHA), preferably Omacor® omega-3 fatty acids is formulated into a single administration or unit dosage.

The dosages of azetidinone-based cholesterol absorption inhibitor with mixtures of omega-3 fatty acids that include eicosapentaenoic acid (EPA) and docosahexaenoic acid (DHA) can be administered together in from 1 to 10 dosages, with the preferred number of dosages from 1 to 4 times a day.

In some preferred embodiments, a soft gelatin capsule is used. The manufacture of soft gelatin capsules is generally known by those of ordinary skill in the art. See, for example, Ebert (1978), “Soft Elastic Gelatin Capsules: A Unique Dosage Form,” Pharmaceutical Technology 1(5), hereby incorporated by reference. In some embodiments, one or more azetidinone-based cholesterol absorption inhibitors, preferably ezetimibe, and/or mixtures of omega-3 fatty acids are contained in the soft gelatin capsule. In certain embodiments, the active ingredients in the soft gelatin capsule are combined with a solubilizer. Solubilizers include surfactants, hydrophilic or hydrophobic solvents, oils or combinations thereof.

One type of solubilizer that may be used is a vitamin E substance. This group of solubilizers includes a substance belonging to the group of α-, β-, γ-, δ-, ζ1-, ζ2- and η-tocopherols, their dl, d and l forms and their structural analogues, such as tocotrienols; the corresponding derivatives, e.g., esters, produced with organic acids; and mixtures thereof. Preferred vitamin E substance solubilizers include tocopherols, tocotrienols and tocopherol derivatives with organic acids such as acetic acid, propionic acid, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid, polyethylene glycol succinate and salicylic acid. Particularly preferred vitamin E substance solubilizers include alpha-tocopherol, alpha-tocopheryl acetate, alpha-tocopheryl acid succinate, alpha-tocopheryl polyethylene glycol 1000 succinate and mixtures thereof.

Another group of solubilizers are monohydric alcohol esters of organic acids. The monohydric alcohol can be, for example, ethanol, isopropanol, t-butanol, a fatty alcohol, phenol, cresol, benzyl alcohol or a cycloalkyl alcohol. The organic acid can be, for example, acetic acid, propionic acid, butyric acid, a fatty acid of 6-22 carbon atoms, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid and salicylic acid. Preferred solubilizers in this group include trialkyl citrates, lower alcohol fatty acid esters and lactones. Preferred trialkyl citrates include triethyl citrate, acetyltriethyl citrate, tributyl citrate, acetyltributyl citrate and mixtures thereof with triethyl citrate being particularly preferred. Particularly preferred lower alcohol fatty acid esters include ethyl oleate, ethyl linoleate, ethyl caprylate, ethyl caprate, isopropyl myristate, isopropyl palmitate and mixtures thereof. Lactones may also serve as a solubilizer. Examples include ε-caprolactone, δ-valerolactone, β-butyrolactone, isomers thereof and mixtures thereof.

The solubilizer may be a nitrogen-containing solvent. Preferred nitrogen-containing solvents include dimethylformamide, dimethylacetamide, N-alkylpyrrolidone, N-hydroxyalkylpyrrolidone, N-alkylpiperidone, N-alkylcaprolactam and mixtures thereof wherein alkyl is a C₁₋₁₂ branched or straight chain alkyl. Particularly preferred nitrogen-containing solvents include N-methyl 2-pyrrolidone, N-ethyl 2-pyrrolidone or a mixture thereof. Alternatively, the nitrogen-containing solvent may be in the form of a polymer such as polyvinylpyrrolidone.

Another group of solubilizers includes phospholipids. Preferred phospholipids include phosphatidylcholine, phosphatidylethanolamine, phosphatidylserine, phosphatidylinositol, lecithins, lysolecithins, lysophosphatidylcholine, polyethylene glycolated phospholipids/lysophospholipids, lecithins/lysolecithins and mixtures thereof.

Another group of preferred solubilizers are glycerol acetates and acetylated glycerol fatty acid esters. Preferred glycerol acetates include acetin, diacetin, triacetin and mixtures thereof, with triacetin being particularly preferred. Preferred acetylated glycerol fatty acid esters include acetylated monoglycerides, acetylated diglycerides and mixtures thereof.

In addition, the solubilizer may be a glycerol fatty acid ester. The fatty acid component is about 6-22 carbon atoms. The glycerol fatty acid ester can be a monoglyceride, diglyceride, triglyceride or mixtures thereof. P referred glycerol fatty acid esters include monoglycerides, diglycerides, medium chain triglycerides with fatty acids having about 6-12 carbons and mixtures thereof. Particularly preferred glycerol fatty acid esters include medium chain monoglycerides with fatty acids having about 6-12 carbons, medium chain diglycerides with fatty acids having about 6-12 carbons and mixtures thereof.

The solubilizer may be a propylene glycol ester. Preferred propylene glycol esters include propylene carbonate, propylene glycol monoacetate, propylene glycol diacetate, propylene glycol fatty acid esters, acetylated propylene glycol fatty acid esters and mixtures thereof. Alternatively, the propylene glycol fatty acid ester may be a propylene glycol fatty acid monoester, propylene glycol fatty acid diester or mixture thereof. The fatty acid has about 6-22 carbon atoms. It is particularly preferred that the propylene glycol ester is propylene glycol monocaprylate (CAPRYOL®). Other preferred propylene glycol esters include propylene glycol dicaprylate, propylene glycol dicaprate, propylene glycol dicaprylate/dicaprate and mixtures thereof.

Another group of solubilizers are ethylene glycol esters. Ethylene glycol esters include monoethylene glycol monoacetates, diethylene glycol esters, polyethylene glycol esters and mixtures thereof. Additional examples include ethylene glycol monoacetates, ethylene glycol diacetates, ethylene glycol fatty acid monoesters, ethylene glycol fatty acid diesters, and mixtures thereof. Alternatively, the ethylene glycol ester may be a polyethylene glycol fatty acid monoesters, polyethylene glycol fatty acid diesters or mixtures thereof. Again, the fatty acid component will contain about 6-22 carbon atoms. Particularly preferred ethylene glycol esters are those marketed under the Labrafil® and Labrasol® names.

Polyoxyethylene-sorbitan-fatty acid esters (also called polysorbates), e.g. of from 4 to 25 alkylene moieties, for example mono- and tri-lauryl, palmityl, stearyl and oleyl esters of the type known and commercially available under the trade name Tween® are also suitable as surfactants.

Hydrophilic solvents which may be used include an alcohol, e.g. a water miscible alcohol, e.g. absolute ethanol, or glycerol. Other alcohols include glycols, e.g. any glycol obtainable from an oxide such as ethylene oxide, e.g. 1,2-propylene glycol. Other examples are polyols, e.g. a polyalkylene glycol, e.g. poly(C₂₋₃)alkylene glycol. A typical example is a polyethylene glycol. Alternatively the hydrophilic component may preferably comprise an N-alkylpyrolidone, e.g. N—(C₁₋₁₄alkyl)pyrolidone, e.g. N-methylpyrolidone, tri(C₁₋₄alkyl)citrate, e.g. triethylcitrate, dimethylisosorbide, (C₅-C₁₃)alkanoic acid, e.g. caprylic acid or propylene carbonate.

The hydrophilic solvent may comprise a main or sole component, e.g. an alcohol, e.g. C1-4-alcohol, e.g. ethanol, or alternatively a co-component, e.g. which may be selected from partial lower ethers or lower alkanols. Preferred partial ethers are, for example, Transcutol® (which has the formula C₂H₅—[O—(CH₂)₂]₂—OH), Glycofurol® (also known as tetrahydrofurfuryl alcohol polyethylene glycol ether), or lower alkanols such as ethanol.

The combination product of one or more azetidinone-based cholesterol absorption inhibitors and concentrated omega-3 fatty acids is aided by the solubility of the one or more azetidinone-based cholesterol absorption inhibitors in the omega-3 fatty acid oil. In some embodiments of the present invention a pharmaceutical composition in unit dosage form comprises an essentially homogeneous solution comprising one or more azetidinone-based cholesterol absorption inhibitors essentially dissolved in solvent system comprising natural or synthetic omega-3 fatty acids or pharmaceutically acceptable esters, derivatives, conjugates, precursors or salts thereof, or mixtures thereof, wherein less than about 10% of the one or more azetidinone-based cholesterol absorption inhibitors is undissolved in the solvent system. The one or more azetidinone-based cholesterol absorption inhibitors are substantially dissolved in the omega-3 fatty acid oil to provide a substantially homogeneous composition. Preferably, this aspect of the present invention does not include high amounts of solubilizers to dissolve the one or more azetidinone-based cholesterol absorption inhibitors. Preferably, the one or more azetidinone-based cholesterol absorption inhibitors are contained in the pharmaceutical composition without the use of large amounts of solubilizers (other than the omega-3 fatty acids), and is substantially dissolved (i.e., less than 10%, preferably less than 5% remains undissolved in the solvent system).

In a preferred embodiment, the one or more azetidinone-based cholesterol absorption inhibitors are completely dissolved. In preferred embodiments, if present at all, solubilizers other than the omega-3 fatty acids are present in amounts of 50% or less w/w based on the total weight of the solvent system in the dosage form, preferably 40% or less, more preferably 30% or less, even more preferably 20% or less, still more preferably 10% or less and most preferably 5% or less. In some embodiments, the solvent system contains no solubilizers other than the omega-3 fatty acids. As used herein, “solvent system” includes the omega-3 fatty acids, generally in the form of an oil. In other preferred embodiments, the weight ratio of omega-3 fatty acids to other solubilizer(s) is at least 0.5 to 1, more preferably at least 1 to 1, even more preferably at least 5 to 1, and most preferably at least 10 to 1.

In preferred embodiments, omega-3 fatty acids are present in amounts of at least 30% w/w based on the total weight of the solvent system in the dosage form, more preferably at least 40%, even more preferably at least 50%, and most preferably at least 60%. In certain embodiments, the amount can be at least 70%, at least 80% or at least 90%.

Dosage forms including the essentially homogenous solution should be stable at room temperature (about 23° C. to 27° C., preferably about 25° C.) and 60% relative humidity for a period of at least one month, preferably at least six months, more preferably at least one year, and most preferably at least two years. By “stable”, applicants mean that the solubilized one or more azetidinone-based cholesterol absorption inhibitors should not precipitate out of solution to any appreciable degree, for example, in amounts of less than 10%, preferably less than 5%.

In addition, dosage forms including the essentially homogenous solution should preserve the one or more azetidinone-based cholesterol absorption inhibitors from degradation. Some embodiments include unit dosage forms of one or more azetidinone-based cholesterol absorption inhibitors and omega-3 fatty acids in which at least 90% of the initial amount of one or more azetidinone-based cholesterol absorption inhibitors in the dosage form at an initial measurement time (t₀) should be maintained after one month storage at room temperature and 60% relative humidity.

The combination product may be manufactured by any method known by those of ordinary skill in the art, by combining the azetidinone-based cholesterol absorption inhibitor(s) with the omega-3 fatty acid(s), and optionally with hydrophilic solvent(s), surfactant(s), other solubilizing agents, and/or other excipients.

Other embodiments of the present invention are directed to suspensions of one or more azetidinone-based cholesterol absorption inhibitors in omega-3 fatty acids. In some embodiments, the suspensions comprise solid crystalline particles, solid amorphous particles, or mixtures thereof of one or more azetidinone-based cholesterol absorption inhibitors in omega-3 fatty acids. Other embodiments include pharmaceutical compositions comprising suspensions of one or more azetidinone-based cholesterol absorption inhibitors in omega-3 fatty acids where a portion of the one or more azetidinone-based cholesterol absorption inhibitors is solubilized in the omega-3 fatty acids or in another component of the composition. For example, in some embodiments, the present invention provides a pharmaceutical composition comprising omega-3 fatty acids and one or more azetidinone-based cholesterol absorption inhibitors, wherein about 1-15% of one or more azetidinone-based cholesterol absorption inhibitors by weight are in solution while the remaining one or more azetidinone-based cholesterol absorption inhibitors are present in suspension.

In other embodiments, the present invention provides a pharmaceutical composition comprising omega-3 fatty acids and one or more azetidinone-based cholesterol absorption inhibitors, wherein at least about 80%, preferably about 85%, more preferably about 90%, even more preferably about 95%, and most preferably about 99%, of the one or more azetidinone-based cholesterol absorption inhibitors by weight are present as solid particles in suspension.

Another embodiment of the present invention is directed to a soft gelatin capsule coated with one or more azetidinone-based cholesterol absorption inhibitors. In such an embodiment, at least one coating applied to the outside of the soft gelatin capsule comprises the one or more azetidinone-based cholesterol absorption inhibitors and a coating material, such as a film forming material and/or binder, and optionally other conventional additives such as lubricants, fillers and antiadherents. Preferred coating materials will include antioxidants, solubilizers, chelating agents and/or absorption enhancers. Surfactants may act as both solubilizers and absorption enhancers.

The coating(s) may be applied by any conventional technique such as pan coating, fluid bed coating or spray coating. The coating(s) may be applied as a suspension, spray, dust or powder. The coating(s) may be formulated for immediate release, delayed/enteric release or sustained release of the second API in accordance with methods well known in the art. Conventional coating techniques are described, e.g., in Remington's Pharmaceutical Sciences, 18th Ed. (1990), hereby incorporated by reference.

An immediate release coating is commonly used to improve product elegance as well as for a moisture barrier, and taste and odor masking. Rapid breakdown of the film in gastric media is important, leading to effective disintegration and dissolution. Eudragit RD100 (Rohm) is an example of such a coating. It is a combination of a water insoluble cationic methacrylate copolymer with a water soluble cellulose ether. In powder form, it is readily dispensable into an easily sprayable suspension that dries to leave a smooth film. Such films rapidly disintegrate in aqueous media at a rate that is independent of pH and film thickness.

A protective coating layer (i.e., seal coat) may be applied, if desired, by conventional coating techniques such as pan coating or fluid bed coating using solutions of polymers in water or suitable organic solvents or by using aqueous polymer dispersions. Suitable materials for the protective layer include cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions and the like. The protective coating layer may include antioxidants, chelating agents, colors or dyes.

The enteric coating layer may be applied onto the cores with or without seal coating by conventional coating techniques, such as pan coating or fluid bed coating using solutions of polymers in water or suitable organic solvents or by using aqueous polymer dispersions. All commercially available pH-sensitive polymers are included. The pharmaceutical active is not released in the acidic stomach environment of approximately below pH 4.5, but not limited to this value. The pharmaceutical active should become available when the pH-sensitive layer dissolves at the greater pH; after a certain delayed time; or after the unit passes through the stomach. The preferred delay time is in the range of two to six hours.

Enteric polymers include cellulose acetate phthalate, Cellulose acetate trimellitate, hydroxypropyl methylcellulose phthalate, polyvinyl acetate phthalate, carboxymethylethylcellulose, co-polymerized methacrylic acid/methacrylic acid methyl esters such as, for instance, materials known under the trade name EUDRAGIT L12.5, L100, or EUDRAGIT S12.5, S100 or similar compounds used to obtain enteric coatings. Aqueous colloidal polymer dispersions or re-dispersions can be also applied, e.g. EUDRAGIT L 30D-55, EUDRAGIT L100-55, EUDRAGIT S100, EUDRAGIT preparation 4110D (Rohm Pharma); AQUATERIC, AQUACOAT CPD 30 (FMC); KOLLICOAT MAE 30D and 30DP (BASF); EASTACRYL 30D (Eastman Chemical).

A sustained release film coat may include a water insoluble material such as a wax or a wax-like substance, fatty alcohols, shellac, zein, hydrogenated vegetable oils, water insoluble celluloses, polymers of acrylic and/or methacrylic acid, and any other slowly digestible or dispersible solids known in the art. The solvent for the hydrophobic coating material may be organic or aqueous. Preferably, the hydrophobic polymer is selected from (i) a water insoluble cellulosic polymer, such as an alkylcellulose, preferably ethylcellulose; (ii) an acrylic polymer; or (iii) mixtures thereof. In other preferred embodiments of the present invention, the hydrophobic material comprising the controlled release coating is an acrylic polymer. Any acrylic polymer which is pharmaceutically acceptable can be used for the purposes of the present invention. The acrylic polymers may be cationic, anionic or non-ionic polymers and may be acrylates, methacrylates, formed of methacrylic acid or methacrylic acid esters. Examples of suitable acrylic polymers include but are not limited to acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamine copolymer, poly(methyl methacrylate), poly(methacrylic acid) (an hydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), and glycidyl methacrylate copolymers.

A barrier coat may be included between an outer coat and the soft gelatin shell. The barrier coat may be comprised of an enteric/delayed release coat (as above), or a barrier (non-functional) layer, which serves as a protective coat to prevent leaching from the shell to the outer API component, or vice versa.

In one embodiment of the invention, an azetidinone-based cholesterol absorption inhibitor, preferably ezetimibe, with mixtures of omega-3 fatty acids are split into first and second portions, with one portion disposed on a coating, and the second portion disposed in the soft gelatin capsule. The dosage form is provided with a lag time between the administration of the first portion and the administration of the second portion, e.g., by an enteric coating provided as a barrier layer. In other embodiments, there is an immediate release of the first portion, followed by a delayed or sustained release of the second portion. In further embodiments, there is a delayed release of the first portion, followed by a bolus of the second portion.

While coating technology is used extensively in the pharmaceutical industry, e.g. for the application of functional or non-functional coats to single dosage forms and for the deposition of APIs onto sugar beads, there are several challenges which can be encountered during coating of soft gelatin capsules. These challenges are often attributed to the properties of gelatin and the dosage form. Soft gelatin capsules generally contain a medicament dissolved or dispersed in oils or hydrophilic liquids (fill liquid). The inherent flexibility of the soft gelatin capsule is due to the presence of plasticizers and residual moisture in the capsule shell. Thus, the soft gelatin capsule is a more dynamic system than conventional tablets or hard gelatin capsules. Atmospheric moisture may permeate into the capsule shell or into the fill liquid. The drug or fill liquid may migrate into the capsule shell, while the plasticizer or residual water gelatin can potentially migrate into the fill liquid. Volatile components in soft gelatin capsules may escape into the atmosphere.

As noted above, polymeric coatings are generally applied as aqueous-based solutions, organic-based solutions or dispersions, in which polymer-containing droplets are atomized with air and sprayed onto the substrate. Heat may be added to the coating equipment to facilitate evaporation of the solvent and film formation. In the case of soft gelatin capsules, the processing parameters of spray rate and bed temperature must be controlled. Because gelatin is soluble in water, spraying an aqueous-based polymeric material at a high rate could lead to solubilization of the gelatin and capsule agglomeration. A high bed temperature may result in the evaporation of residual water from the capsule shell, causing the capsule to become brittle. Therefore, the present invention comprises a method of coating soft gelatin capsules in which these consequences are avoided.

In addition, the deposition of a low dose of one or more azetidinone-based cholesterol absorption inhibitors onto the surface of the soft gelatin capsules with high degree of accuracy could be affected by several factors. The accuracy of deposition needs to be demonstrated by evaluating coating uniformity which includes the mass variance of the coated capsules and the variance of the content of the coated one or more azetidinone-based cholesterol absorption inhibitors.

The present invention provides for a method of coating a soft gelatin capsule comprising mixtures of omega-3 fatty acids, with a coating comprising a coating material and one or more azetidinone-based cholesterol absorption inhibitors, the method comprising controlling the rate of coating deposition on the soft gelatin capsule and controlling the temperature during the coating process to produce a physically and chemically stable coated soft gelatin capsule.

In other embodiments, the coating of the present invention may also be applied onto a hard gelatin capsule or a tablet. The hard gelatin capsule may contain, instead of liquid, powder, beads or microtablets (e.g., similar system to U.S. Pat. No. 5,681,588, incorporated herein by reference).

Yet other embodiments of the present invention include a unit dosage of one or more azetidinone-based cholesterol absorption inhibitors and omega-3 fatty acids in which at least 90% of the initial amount of one or more azetidinone-based cholesterol absorption inhibitors in the dosage form at an initial measurement time (t₀) should be maintained after one month storage at room temperature and 60% relative humidity.

In some embodiments, the combination of the present invention allow for improved effectiveness of each active ingredient, with one or both administered as a conventional full-strength dose, as compared to the formulations in the prior art. In other embodiments, the formulations of the present invention may allow for reduced dosages of an azetidinone-based cholesterol absorption inhibitor and/or omega-3 fatty acids, as compared to the formulations in the prior art, while still maintaining or even improving upon the effectiveness of each active ingredient.

The present combination of azetidinone-based cholesterol absorption inhibitor with mixtures of omega-3 fatty acids that include eicosapentaenoic acid (EPA) and docosahexaenoic acid (DHA) may allow for a greater effect than any expected combined or additive effect of the two drugs alone. Thus, the combined treatment of the two active ingredients, separately or through the novel combination product of the present invention, may cause an unexpected increase in effect of the active ingredients that allows increased effectiveness with standard dosages or maintained effectiveness with reduced dosages of the two active ingredients. It is well accepted in practice that an improved bioavailability or effectiveness of a drug or other active ingredient allows for an appropriate reduction in the daily dosage amount. Any undesirable side effects may also be reduced as a result of the lower dosage amount and the reduction in excipients (e.g., surfactants).

The utilization of a single administration of a combination of a an azetidinone-based cholesterol absorption inhibitor with mixtures of omega-3 fatty acids that include eicosapentaenoic acid (EPA) and docosahexaenoic acid (DHA) overcomes the limitations of the prior art by improving the efficacy of an azetidinone-based cholesterol absorption inhibitor with mixtures of omega-3 fatty acids that include eicosapentaenoic acid (EPA) and docosahexaenoic acid (DHA), and may allow for a treatment with improved effectiveness and fewer excipients than in the prior art.

All references cited herein are incorporated by reference in their entirety.

1. A pharmaceutical composition comprising:
a. a unit dosage form comprising natural or synthetic omega-3 fatty acids or pharmaceutically acceptable esters, derivatives, conjugates, precursors or salts thereof, or mixtures thereof and optionally a solubilizer, and
b. one or more outer coatings on the unit dosage form, wherein at least one outer coating comprises one or more azetidinone-based cholesterol absorption inhibitors,
c. optionally one or more barrier coatings between the unit dosage form and the one or more outer coatings, and
d. optionally a seal coating on the unit dosage form.

2. The pharmaceutical composition of claim 1, wherein one or more outer coatings is formulated for immediate release, delayed/enteric release or sustained release of the one or more azetidinone-based cholesterol absorption inhibitors.

3. The pharmaceutical composition of claim 1, wherein one or more barrier coatings is formulated for enteric/delayed release of the natural or synthetic omega-3 fatty acids or pharmaceutically acceptable esters, derivatives, conjugates, precursors or salts thereof, or mixtures thereof, or as a nonfunctional protective layer.

4. The pharmaceutical composition of claim 1, wherein the unit dosage form is a soft gelatin capsule, a hard gelatin capsule, or a tablet.

5. The pharmaceutical composition of claim 1, wherein the one or more azetidinone-based cholesterol absorption inhibitors is ezetimibe.

6. The pharmaceutical composition of claim 1, wherein the omega-3 fatty acids contain at least about 70% EPA and DHA.

7. The pharmaceutical composition of claim 1, comprising about 0.1 g to about 10 g omega-3 fatty acids or pharmaceutically acceptable esters, derivatives, conjugates, precursors or salts thereof, or mixtures thereof.

8. The pharmaceutical composition of claim 1, comprising from about 2 mg to about 150 mg of one or more azetidinone-based cholesterol absorption inhibitors.

9. The pharmaceutical composition of claim 1, wherein the at least one outer coating comprising one or more azetidinone-based cholesterol absorption inhibitors is sprayed onto the unit dosage form while controlling the rate of coating deposition and controlling the temperature during the coating process to produce a physically and chemically stable coated unit dosage form.

10. A pharmaceutical composition in unit dosage form, comprising a heterogeneous suspension or an essentially homogenous solution of one or more azetidinone-based cholesterol absorption inhibitors in a solvent system comprising natural or synthetic omega-3 fatty acids or pharmaceutically acceptable esters, derivatives, conjugates, precursors or salts thereof, or mixtures thereof.

11. The pharmaceutical composition of claim 10, wherein the omega-3 fatty acids contain at least about 70% EPA and DHA.

12. The pharmaceutical composition of claim 10, wherein the pharmaceutical composition comprises the heterogeneous suspension.

13. The pharmaceutical composition of claim 12, wherein at least about 80% of the one or more azetidinone-based cholesterol absorption inhibitors are present as solid particles in the suspension.

14. The pharmaceutical composition of claim 10, wherein the pharmaceutical composition comprises the essentially homogeneous solution.

15. The pharmaceutical composition of claim 14, wherein less than about 10% of the one or more azetidinone-based cholesterol absorption inhibitors is undissolved in the solvent system.

16. The pharmaceutical composition of claim 14, wherein the solvent system further comprises at least one solubilizer in an amount of 50% or less w/w based on the total weight of the solvent system.

17. The pharmaceutical composition of claim 14, wherein no more than 10% of the dissolved one or more azetidinone-based cholesterol absorption inhibitors precipitates out of the essentially homogenous solution when the pharmaceutical composition is stored at room temperature and 60% relative humidity for a period of at least one month.

18. A method of treating a subject having one or more conditions selected from the group consisting of dyslipidemia or related conditions, renal disease, hypercholesterolemia, hypertension, elevated total cholesterol (total-C), elevated low density lipoprotein cholesterol (LDL-C), elevated apolipoprotein (Apo B), low high density lipoprotein cholesterol (HDL-C), elevated sitosterol, elevated campesterol, sitosterolemia, cholesterol-associated benign, malignant tumors, coronary heart disease, vascular disease, and related disorders, events, and/or symptoms, hypertriglyceridemia, artherosclerotic disease and related conditions, patients in need of the prevention or reduction of cardiovascular and vascular events, and the reduction of triglyceride levels, insulin resistance, fasting glucose levels and postprandial glucose levels, comprising administering to the subject an effective amount of one or more azetidinone-based cholesterol absorption inhibitors and natural or synthetic omega-3 fatty acids or pharmaceutically acceptable esters, derivatives, conjugates, precursors or salts thereof, or mixtures thereof.

19. The method of claim 18, wherein the subject has mixed dyslipidemia, combined hyperlipidemia, or high non-HDL-C.
